SEQUENCE LISTING (6 sequences)

SEQ ID NO: 1 - DNA, 1437 bp, Homo sapiens
atgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtctga

SEQ ID NO: 2 - protein, 478 aa, Homo sapiens
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIV

SEQ ID NO: 3 - DNA, 1422 bp, Homo sapiens
atgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtctga

SEQ ID NO: 4 - protein, 473 aa, Homo sapiens
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIV

SEQ ID NO: 5 - DNA, 4586 bp, Homo sapiens
gaattccgccctcgccgcccgcggcgccccgagcgctttgtgagcagatgcggagccgagtggagggcgcgagccagatgcggggcgacagctgacttgctgagaggaggcggggaggcgcggagcgcgcgtgtggtccttgcgccgctgacttctccactggttcctgggcaccgaaagataaacctctcataatgaaggcccccgctgtgcttgcacctggcatcctcgtgctcctgtttaccttggtgcagaggagcaatggggagtgtaaagaggcactagcaaagtccgagatgaatgtgaatatgaagtatcagcttcccaacttcaccgcggaaacacccatccagaatgtcattctacatgagcatcacattttccttggtgccactaactacatttatgttttaaatgaggaagaccttcagaaggttgctgagtacaagactgggcctgtgctggaacacccagattgtttcccatgtcaggactgcagcagcaaagccaatttatcaggaggtgtttggaaagataacatcaacatggctctagttgtcgacacctactatgatgatcaactcattagctgtggcagcgtcaacagagggacctgccagcgacatgtctttccccacaatcatactgctgacatacagtcggaggttcactgcatattctccccacagatagaagagcccagccagtgtcctgactgtgtggtgagcgccctgggagccaaagtcctttcatctgtaaaggaccggttcatcaacttctttgtaggcaataccataaattcttcttatttcccagatcatccattgcattcgatatcagtgagaaggctaaaggaaacgaaagatggttttatgtttttgacggaccagtcctacattgatgttttacctgagttcagagattcttaccccattaagtatgtccatgcctttgaaagcaacaattttatttacttcttgacggtccaaagggaaactctagatgctcagacttttcacacaagaataatcaggttctgttccataaactctggattgcattcctacatggaaatgcctctggagtgtattctcacagaaaagagaaaaaagagatccacaaagaaggaagtgtttaatatacttcaggctgcgtatgtcagcaagcctggggcccagcttgctagacaaataggagccagcctgaatgatgacattcttttcggggtgttcgcacaaagcaagccagattctgccgaaccaatggatcgatctgccatgtgtgcattccctatcaaatatgtcaacgacttcttcaacaagatcgtcaacaaaaacaatgtgagatgtctccagcatttttacggacccaatcatgagcactgctttaataggacacttctgagaaattcatcaggctgtgaagcgcgccgtgatgaatatcgaacagagtttaccacagctttgcagcgcgttgacttattcatgggtcaattcagcgaagtcctcttaacatctatatccaccttcattaaaggagacctcaccatagctaatcttgggacatcagagggtcgcttcatgcaggttgtggtttctcgatcaggaccatcaacccctcatgtgaattttctcctggactcccatccagtgtctccagaagtgattgtggagcatacattaaaccaaaatggctacacactggttatcactgggaagaagatcacgaagatcccattgaatggcttgggctgcagacatttccagtcctgcagtcaatgcctctctgccccaccctttgttcagtgtggctggtgccacgacaaatgtgtgcgatcggaggaatgcctgagcgggacatggactcaacagatctgtctgcctgcaatctacaaggttttcccaaatagtgcaccccttgaaggagggacaaggctgaccatatgtggctgggactttggatttcggaggaataataaatttgatttaaagaaaactagagttctccttggaaatgagagctgcaccttgactttaagtgagagcacgatgaatacattgaaatgcacagttggtcctgccatgaataagcatttcaatatgtccataattatttcaaatggccacgggacaacacaatacagtacattctcctatgtggatcctgtaataacaagtatttcgccgaaatacggtcctatggctggtggcactttacttactttaactggaaattacctaaacagtgggaattctagacacatttcaattggtggaaaaacatgtactttaaaaagtgtgtcaaacagtattcttgaatgttataccccagcccaaaccatttcaactgagtttgctgttaaattgaaaattgacttagccaaccgagagacaagcatcttcagttaccgtgaagatcccattgtctatgaaattcatccaaccaaatcttttattagtggtgggagcacaataacaggtgttgggaaaaacctgaattcagttagtgtcccgagaatggtcataaatgtgcatgaagcaggaaggaactttacagtggcatgtcaacatcgctctaattcagagataatctgttgtaccactccttccctgcaacagctgaatctgcaactccccctgaaaaccaaagcctttttcatgttagatgggatcctttccaaatactttgatctcatttatgtacataatcctgtgtttaagccttttgaaaagccagtgatgatctcaatgggcaatgaaaatgtactggaaattaagggaaatgatattgaccctgaagcagttaaaggtgaagtgttaaaagttggaaataagagctgtgagaatatacacttacattctgaagccgttttatgcacggtccccaatgacctgctgaaattgaacagcgagctaaatatagagtggaagcaagcaatttcttcaaccgtccttggaaaagtaatagttcaaccagatcagaatttcacaggattgattgctggtgttgtctcaatatcaacagcactgttattactacttgggtttttcctgtggctgaaaaagagaaagcaaattaaagatctgggcagtgaattagttcgctacgatgcaagagtacacactcctcatttggataggcttgtaagtgcccgaagtgtaagcccaactacagaaatggtttcaaatgaatctgtagactaccgagctacttttccagaagatcagtttcctaattcatctcagaacggttcatgccgacaagtgcagtatcctctgacagacatgtcccccatcctaactagtggggactctgatatatccagtccattactgcaaaatactgtccacattgacctcagtgctctaaatccagagctggtccaggcagtgcagcatgtagtgattgggcccagtagcctgattgtgcatttcaatgaagtcataggaagagggcattttggttgtgtatatcatgggactttgttggacaatgatggcaagaaaattcactgtgctgtgaaatccttgaacagaatcactgacataggagaagtttcccaatttctgaccgagggaatcatcatgaaagattttagtcatcccaatgtcctctcgctcctgggaatctgcctgcgaagtgaagggtctccgctggtggtcctaccatacatgaaacatggagatcttcgaaatttcattcgaaatgagactcataatccaactgtaaaagatcttattggctttggtcttcaagtagccaaaggcatgaaatatcttgcaagcaaaaagtttgtccacagagacttggctgcaagaaactgtatgctggatgaaaaattcacagtcaaggttgctgattttggtcttgccagagacatgtatgataaagaatactatagtgtacacaacaaaacaggtgcaaagctgccagtgaagtggatggctttggaaagtctgcaaactcaaaagtttaccaccaagtcagatgtgtggtcctttggcgtcgtcctctgggagctgatgacaagaggagccccaccttatcctgacgtaaacacctttgatataactgtttacttgttgcaagggagaagactcctacaacccgaatactgcccagaccccttatatgaagtaatgctaaaatgctggcaccctaaagccgaaatgcgcccatccttttctgaactggtgtcccggatatcagcgatcttctctactttcattggggagcactatgtccatgtgaacgctacttatgtgaacgtaaaatgtgtcgctccgtatccttctctgttgtcatcagaagataacgctgatgatgaggtggacacacgaccagcctccttctgggagacatcatagtgctagtactatgtcaaagcaacagtccacactttgtccaatggttttttcactgcctgacctttaaaaggccatcgatattctttgctccttgccaaattgcactattaataggacttgtattgttatttaaattactggattctaaggaatttcttatctgacagagcatcagaaccagaggcttggtcccacaggccagggaccaatgcgctgcag

SEQ ID NO: 6 - protein, 1390 aa, Homo sapiens [sequence omitted]

(19) United States
(12) Patent Application Publication
(10) Pub. No.: US 2012/0064090 A1
(43) Pub. Date: Mar. 15, 2012

(54) THERAPEUTIC AGENT FOR CANCER HAVING REDUCED SENSITIVITY TO MOLECULAR TARGET DRUG AND PHARMACEUTICAL COMPOSITION FOR ENHANCING SENSITIVITY TO MOLECULAR TARGET DRUG

(75) Inventors: Seiji Yano, Ishikawa (JP); Kunio Matsumoto, Ishikawa (JP)

(73) Assignee: KRINGLE PHARMA INC., Osaka (JP)

(21) Appl. No.: 13/258,718

(22) PCT Filed: Oct. 9, 2009
(86) PCT No.: PCT/JP2009/067630

§ 371 Date: Nov. 7, 2011

(30) Foreign Application Priority Data
Mar. 27, 2009 (JP) 2009-078621

(51) Int. Cl. A61K 39/395; C07K 14/47; A61K 31/496; A61P 35/00; A61P 35/02; C07D 403/14; C07K 16/22; A61K 38/17

(52) U.S. Cl. 424/158.1; 530/389.2; 530/350; 514/19.3; 514/19.4; 514/19.5; 514/19.6; 544/373; 514/254.09

(57) ABSTRACT

Provided are a pharmaceutical composition which enhances the sensitivity of a cancer to a molecular target drug, such as gefitinib and erlotinib, wherein the cancer has resistance to the molecular target drug, and a cancer therapeutic agent effective against a cancer having resistance to a molecular target drug, such as gefitinib and erlotinib. The pharmaceutical composition comprising an HGF-MET receptor pathway inhibitor enhances the sensitivity of a cancer to a molecular target drug, such as gefitinib and erlotinib, even though the cancer has resistance to the molecular target drug. The cancer therapeutic agent comprising a molecular target drug in combination with an HGF-MET receptor pathway inhibitor is effective against a cancer having resistance to the molecular target drug.

### TECHNICAL FIELD

The present invention relates to a pharmaceutical composition and a therapeutic agent each of which enhances the sensitivity of a cancer to a molecular target drug, wherein the cancer is a target of treatment with the molecular target drug but is refractory to the molecular target drug or less sensitive to the molecular target drug.

### BACKGROUND ART

Lung cancer is a malignant tumor and the number one cause of death in our country, and it is urgent to establish an effective therapeutic method therefor. In recent years, epidermal growth factor receptor (hereinafter referred to as “EGFR”) tyrosine kinase inhibitors (gefitinib, erlotinib, etc.), which are molecular target drugs, have been approved as a drug for lung cancer. EGFR tyrosine kinase inhibitors are remarkably effective in lung cancer patients who have EGFR gene mutations and who are nonsmokers, and thus considered to be a specific medicine for lung cancer. However, the most patients of effective case develop acquired resistance and recurrence in about one year, and 25 to 30% of patients with lung adenocarcinoma harboring EGFR gene mutations are intrinsically resistant to gefitinib. Therefore, overcoming gefitinib and erlotinib resistance in lung adenocarcinoma harboring EGFR gene mutations is a vital problem to be solved from the clinical viewpoint.

Regarding the resistance to EGFR tyrosine kinase inhibitors in lung cancer, Non Patent Literature 1 has a description: amplification of MET gene was observed in lung cancer cells with acquired gefitinib resistance, and proliferation of the gefitinib-resistant lung cancer cells was inhibited by combined use of a MET inhibitor and gefitinib. The present inventors reported that induction of gefitinib resistance in scirrhous gastric carcinoma cells by interaction with fibroblasts was inhibited by combined use of NK4 gene therapy and gefitinib (see Non Patent Literature 2).

### CITATION LIST

### Non Patent Literature

### Non Patent Literature 1:

Engelman J. A. et al., Science: 316, 1039-1043 (2007)

### Non Patent Literature 2:

Namiki Y. et al., Int. J. Cancer: 118, 1545-1555 (2006)

### SUMMARY OF INVENTION

### Technical Problem

An object of the present invention is to provide a pharmaceutical composition which enhances the sensitivity of a cancer to a molecular target drug, such as gefitinib and erlotinib, wherein the cancer has resistance to the molecular target drug, and to provide a cancer therapeutic agent effective against a cancer having resistance to a molecular target drug, such as gefitinib and erlotinib.

### Solution to Problem

The present invention includes the following as a solution to the above-mentioned problems.

[1] A pharmaceutical composition comprising an HGF-MET receptor pathway inhibitor, which enhances the sensitivity of a cancer to a molecular target drug, wherein the cancer is a target of treatment with the molecular target drug but is refractory to the molecular target drug or less sensitive to the molecular target drug.
[2] The pharmaceutical composition according to the above [1], wherein the cancer refractory to the molecular target drug or less sensitive to the molecular target drug is not accompanied by amplification of MET receptor gene.
[3] The pharmaceutical composition according to the above [1] or [2], wherein the molecular target drug is an EGFR tyrosine kinase inhibitor.
[4] The pharmaceutical composition according to the above [3], wherein the EGFR tyrosine kinase inhibitor is a reversible EGFR tyrosine kinase inhibitor.
[5] The pharmaceutical composition according to the above [3], wherein the EGFR tyrosine kinase inhibitor is an irreversible EGFR tyrosine kinase inhibitor.
[6] The pharmaceutical composition according to any one of the above [1] to [5], wherein the HGF-MET receptor pathway inhibitor is one or more kinds selected from the group consisting of an anti-HGF neutralizing antibody, NK4, a MET receptor tyrosine kinase inhibitor, an anti-MET receptor antibody, a MET receptor expression inhibitor and a protein having an HGF-binding domain of a MET receptor extracellular region.
[7] The pharmaceutical composition according to any one of the above [1] to [6], wherein the cancer being a target of treatment with the molecular target drug is lung cancer, breast cancer, colon cancer, prostate cancer, brain tumor, pancreatic cancer, gallbladder cancer, renal cancer, chronic myelogenous leukemia, gastrointestinal stromal tumor, esophageal cancer, head-and-neck tumor or gastric cancer.
[8] A cancer therapeutic agent comprising a molecular target drug in combination with an HGF-MET receptor pathway inhibitor, wherein the cancer is a target of treatment with the molecular target drug but is refractory to the molecular target drug or less sensitive to the molecular target drug.
[9] The cancer therapeutic agent according to the above [8], wherein the cancer refractory to the molecular target drug or less sensitive to the molecular target drug is not accompanied by amplification of MET receptor gene.
[10] The cancer therapeutic agent according to the above [8] or [9], wherein the molecular target drug is an EGFR tyrosine kinase inhibitor.
[11] The cancer therapeutic agent according to the above [10], wherein the EGFR tyrosine kinase inhibitor is a reversible EGFR tyrosine kinase inhibitor.
[12] The cancer therapeutic agent according to the above [10], wherein the EGFR tyrosine kinase inhibitor is an irreversible EGFR tyrosine kinase inhibitor.
[13] The cancer therapeutic agent according to any one of the above [8] to [12], wherein the HGF-MET receptor pathway inhibitor is one or more kinds selected from the group consisting of an anti-HGF neutralizing antibody, NK4, a MET receptor tyrosine kinase inhibitor, an anti-MET receptor antibody, a MET receptor expression inhibitor and a protein having an HGF-binding domain of a MET receptor extracellular region.
[14] The cancer therapeutic agent according to any one of the above [8] to [13], wherein the cancer being a target of treatment with the molecular target drug is lung cancer, breast cancer, colon cancer, prostate cancer, brain tumor, pancreatic cancer, gallbladder cancer, renal cancer, chronic myelogenous leukemia, gastrointestinal stromal tumor, esophageal cancer, head-and-neck tumor or gastric cancer.

### ADVANTAGEOUS EFFECTS OF INVENTION

The present invention can provide a pharmaceutical composition which enhances the sensitivity of a cancer to a molecular target drug, such as gefitinib and erlotinib, wherein the cancer has resistance to the molecular target drug. The present invention can also provide a cancer therapeutic agent effective against a cancer having resistance to a molecular target drug, such as gefitinib and erlotinib. The present invention is beneficial for cancer patients, and its social significance is extremely great.

### BRIEF DESCRIPTION OF DRAWINGS

FIG. 1(a) shows the growth rates of lung cancer cell PC-9 after 72-hour culture in culture media containing various concentrations of gefitinib and HGF.

FIG. 1(b) shows the growth rates of lung cancer cell HCC827 after 72-hour culture in culture media containing various concentrations of gefitinib and HGF.

FIG. 2 shows the growth rates of lung cancer cell HCC827 after 72-hour culture in culture media with or without gefitinib in the presence of HGF pretreated with the anti-HGF neutralizing antibody or the control IgG.

FIG. 3(a) shows the growth rates of lung cancer cell PC-9 after 72-hour culture in culture media containing various growth factors and different concentrations of gefitinib.

FIG. 3(b) shows the growth rates of lung cancer cell HCC827 after 72-hour culture in culture media containing various growth factors and different concentrations of gefitinib.

FIG. 4 shows the effects of gefitinib, the anti-HGF neutralizing antibody or the control IgG on the growth of lung cancer cell PC-9 transfected with the HGF expression vector.

FIG. 5 shows the growth rates of lung cancer cell PC-9 cultured with or without HGF or of lung cancer cell PC-9 cocultured with fibroblast MRC-5 for 72 hours in culture media with or without gefitinib and the anti-HGF neutralizing antibody or the control IgG.

FIG. 6 shows the combined effects of gefitinib and the anti-HGF neutralizing antibody or NK4 on tumor derived from gefitinib-sensitive human lung cancer cells subcutaneously transplanted in the back of SCID mice.

FIG. 7 shows the HGF concentrations measured in tumor derived from gefitinib-sensitive human lung cancer cells subcutaneously transplanted in the back of SCID mice.

FIG. 8 shows the growth rates of lung cancer cell H1975 after 72-hour culture in culture media containing various concentrations of gefitinib or the irreversible EGFR tyrosine kinase inhibitor CL-387,785.

FIG. 9 shows the growth rates of lung cancer cell H1975 after 72-hour culture in culture media containing various concentrations of the irreversible EGFR tyrosine kinase inhibitor CL-387,785 with or without HGF.

FIG. 10 shows the effects of the irreversible EGFR tyrosine kinase inhibitor CL-387,785, HGF, the anti-HGF neutralizing antibody or NK4 on the growth of lung cancer cell H1975.

FIG. 11 shows the effects of gefitinib, HGF, the anti-HGF neutralizing antibody, NK4 or SU11274 on the growth of lung cancer cell PC-9.

FIG. 12 shows the effects of CL-387,785, HGF, the anti-HGF neutralizing antibody, NK4 or SU11274 on the growth of lung cancer cell H1975.

### DESCRIPTION OF EMBODIMENTS

The pharmaceutical composition of the present invention comprises an HGF-MET receptor pathway inhibitor, and enhances the sensitivity of a cancer to a molecular target drug, wherein the cancer is a target of treatment with the molecular target drug but is refractory to the molecular target drug or less sensitive to the molecular target drug. The cancer therapeutic agent of the present invention comprises a molecular target drug in combination with an HGF-MET receptor pathway inhibitor, and is intended for treatment of a cancer which is a target of treatment with the molecular target drug but is refractory to the molecular target drug or less sensitive to the molecular target drug.

The molecular target drug means an agent designed so as to efficiently act on a molecular level target reflecting a nature specific to cancer cells. Examples thereof include proteins such as antibodies, peptides, nucleic acids and low molecular weight compounds. Examples of the molecular target drug of the present invention include, but are not limited to, known molecular target drugs such as gefitinib, erlotinib, imatinib, ibritumomab tiuxetan, gemtuzumab ozogamicin, sunitinib, cetuximab, sorafenib, tamibarotene, trastuzumab, tretinoin, panitumumab, bevacizumab, bortezomib, rituximab, vandetanib, lapatinib, sorafenib and cetuximab. Inter alfa, preferred is an EGFR (Epidermal Growth Factor Receptor) tyrosine kinase inhibitor. EGFR tyrosine kinase inhibitors are medications which exhibit an anticancer effect by inhibition of signal transduction from EGFR expressed on the surface of cancer cells. EGFR tyrosine kinase inhibitors include reversible EGFR tyrosine kinase inhibitors and irreversible EGFR tyrosine kinase inhibitors, and either type of EGFR tyrosine kinase inhibitors is suitable as the molecular target drug of the present invention. Examples of the reversible EGFR tyrosine kinase (including the EGFR family) inhibitor include gefitinib, erlotinib, cetuximab and trastuzumab. Examples of the irreversible EGFR tyrosine kinase inhibitor include EKB569, HKI2721, BIBW2992, PF299804, CL-387,785 and CI-1033.

According to the present invention, preferable examples of the target cancer of treatment with the molecular target drug include, but are not limited, lung cancer, breast cancer, colon cancer, prostate cancer, brain tumor (glioma, glioblastoma, medulloblastoma, etc.), pancreatic cancer, gallbladder cancer, renal cancer, chronic myelogenous leukemia, gastrointestinal stromal tumor, esophageal cancer, head-and-neck tumor and gastric cancer.

The cancer refractory to the molecular target drug is a cancer that is refractory to the molecular target drug due to mutation in a target molecule itself of the molecular target drug; mutation of another molecule in the signaling pathway associated with the target molecule; mutation, overexpression or secondarily-induced activation of another molecule not directly involved in the signaling pathway associated with the target molecule; or the like. Such a cancer includes a cancer having such a nature (drug resistance) prior to the start of treatment with the molecular target drug, and a cancer having such a nature as one acquired during treatment with the molecular target drug. The cancer less sensitive to the molecular target drug is a cancer that has reduced sensitivity to the molecular target drug due to mutation in a target molecule itself of the molecular target drug; mutation of another molecule in the signaling pathway associated with the target molecule; mutation, overexpression or secondarily-induced activation of another molecule not directly involved in the signaling pathway associated with the target molecule; or the like. Such a cancer includes a cancer having such a nature (reduced sensitivity) prior to the start of treatment with the molecular target drug, and a cancer having such a nature as one acquired during treatment with the molecular target drug.

What makes cancers refractory or less sensitive to a molecular target drug is still largely unclear. This will be explained by taking gefitinib as an example. Gefitinib is intended for treatment of non-small-cell lung cancer, and is particularly effective in a non-small-cell lung cancer expressing an EGFR with activating mutation caused by deletion mutation in exon 19 of the EGFR gene or by mutation of Leu to Thr at residue 858. However, it is known that, before or during treatment with gefitinib, new mutation in EGFR (Thr to Met at residue 790 etc.) or amplification of MET receptor gene makes non-small-cell lung cancer refractory or less sensitive to gefitinib. In particular, of the cases of non-small-cell lung cancers with EGFR-activating mutation and reduced sensitivity to gefitinib, amplification of MET receptor gene is observed in about 20% (see Non Patent Literature 1), and new mutation in the amino acid sequence of EGFR (T790M etc.) is observed in about 50%.

The present inventors found that, in non-small-cell lung cancers with EGFR-activating mutation, HGF makes the cancer refractory or less sensitive to gefitinib. This finding revealed that activation of HGF-MET receptor pathway is in part responsible for making such cancers refractory or less sensitive to a molecular target drug. As described above, of the cases of non-small-cell lung cancers with EGFR-activating mutation and acquired resistance to gefitinib, amplification of MET receptor gene is observed in about 20% and new mutation in EGFR is observed in about 50%, but the cause is not yet clarified in the remaining (about 30%). It is also known that about 30% of patients with a non-small-cell lung cancer having EGFR-activating mutation are intrinsically resistant to gefitinib. Activation of HGF-MET receptor pathway found by the present inventors in cancers refractory or less sensitive to a molecular target drug is in part responsible for making cancers refractory or less sensitive to gefitinib without amplification of MET receptor gene or new mutation in EGFR.

In the present invention, “a cancer which is a target of treatment with a molecular target drug but is refractory to the molecular target drug or less sensitive to the molecular target drug” is not particularly limited, but is preferably a cancer not accompanied by amplification of MET receptor gene. Whether or not the cancer is accompanied by amplification of MET receptor gene can be confirmed by, for example, extracting genomic DNA from target cancer cells, performing quantitative PCR by use of appropriate primers for amplification of the MET receptor gene, and comparing the results with those of cells without amplification of MET receptor gene.

In the present invention, “a cancer which is a target of treatment with a molecular target drug but is refractory to the molecular target drug or less sensitive to the molecular target drug” is preferably a cancer refractory or less sensitive to the molecular target drug due to activation of HGF-MET receptor pathway. Particularly preferred is a cancer that is refractory or less sensitive to the molecular target drug due to activation of HGF-MET receptor pathway but not accompanied by amplification of MET receptor gene.

The HGF-MET receptor pathway inhibitor means a substance capable of inhibiting signal transduction from MET receptor, and includes proteins, peptides, nucleic acids and low molecular weight compounds. Specifically, the signal transduction from MET receptor is inhibited by inhibition of binding of HGF to MET receptor through interaction with HGF, inhibition of binding of HGF to MET receptor through interaction with MET receptor, inhibition of signal transduction from MET receptor through interaction with MET receptor, inhibition of MET receptor expression, etc. According to the present invention, preferable examples of the HGF-MET receptor pathway inhibitor include an anti-HGF neutralizing antibody, NK4, a MET receptor tyrosine kinase inhibitor, an anti-MET receptor antibody, a MET receptor expression inhibitor and a protein having an HGF-binding domain of a MET receptor extracellular region.

The anti-HGF neutralizing antibody may be any antibody that binds to HGF and thereby reduces or blocks the activity of HGF. For example, an anti-HGF antibody that binds to HGF and thereby prevents HGF from binding to MET receptor is included. The anti-HGF neutralizing antibody is producible by use of HGF or its fragment as an immunogen according to a known method described later. Whether or not the resulting antibody is a neutralizing antibody can be confirmed by testing neutralizing action of the resulting antibody on the activity of HGF. Specifically, for example, it can be confirmed by testing neutralizing action on HGF-promoted DNA synthesis in primary cultured hepatocytes, or neutralizing action on HGF-induced cell scattering of MDCK canine kidney epithelial cells. The HGF-MET receptor pathway inhibitor of the present invention also includes an anti-HGF humanized monoclonal antibody which is being or will be developed as an antibody drug.

NK4 is a protein having an N-terminal hairpin domain of and four kringle domains of the HGF a chain, and binds to MET receptor and thereby acts as an antagonist of HGF (Date. K et al., FEBS Lett, 420, 1-6 (1997); and Date. K et al., Oncogene, 17, 3045-3054 (1998)). NK4 can be obtained by, for example according to a known recombinant technique, constructing an NK4 expression vector using an NK4-encoding gene, introducing the vector into a suitable host, and collecting and purifying the resulting recombinant NK4 expressed in the host. In addition, gene medicine and gene therapy vectors using an NK4 expression vector prepared by inserting an NK4-encoding gene into a suitable vector are also included in the NK4 as an HGF-MET receptor pathway inhibitor. Examples of the NK4-encoding gene include, but are not limited to, a gene comprising the base sequence of SEQ ID NO: 1 or 3. The NK4 gene comprising the base sequence of SEQ ID NO: 1 encodes an NK4 protein comprising the amino acid sequence of SEQ ID NO: 2, and the NK4 gene comprising the base sequence of SEQ ID NO: 3 encodes an NK4 protein comprising the amino acid sequence of SEQ ID NO: 4.

The MET receptor tyrosine kinase inhibitor may be any substance that inhibits the tyrosine kinase activity of MET receptor, and examples thereof include, but are not limited to, inhibitory agents for the tyrosine kinase activity of MET receptor such as SU11274 (Pfizer), PHA665752 (Pfizer), PF2341066 (Pfizer), XL880 (Exelixis), ARQ197 (ArQule), MK2461 (Merck), MP470 (SuperGen), SGX523 (SGX Pharmaceutical) and JNJ38877605 (Johnson & Johnson). Also, molecules that inhibit the tyrosine kinase activity of MET receptor are included.

The anti-MET receptor antibody may be any antibody that binds to MET receptor and thereby inhibits signal transduction therefrom. For example, an antibody that binds to the HGF-binding site of MET receptor and thereby inhibits HGF from binding to the receptor is included. The anti-MET receptor antibody is producible by use of MET receptor or its fragment as an immunogen according to a known method described later. Whether or not the resulting antibody is an antibody capable of inhibiting the signal transduction can be confirmed by testing neutralizing action of the resulting antibody on the activity of HGF. Specifically, for example, it can be confirmed by testing neutralizing action on HGF-promoted DNA synthesis in primary cultured hepatocytes, or neutralizing action on HGF-induced cell scattering of MDCK canine kidney epithelial cells.

The MET receptor expression inhibitor may be any substance that inhibits expression of MET receptor. For example, siRNA (short interfering RNA), shRNA (short hairpin RNA), antisense oligonucleotide, etc. for MET receptor gene are included. Examples of the MET receptor gene include, but are not limited to, a gene comprising the base sequence of SEQ ID NO: 5. siRNA is a double-stranded RNA of about 20 bases (for example, about 21 to 23 bases) or less in length. Such siRNA, after expressed in cells, can inhibit expression of its target gene (MET receptor gene in the present invention). shRNA is a molecule consisting of about 20 base pairs or more and, as a single-stranded RNA, comprises a palindromic sequence that enables the molecule to form a double-stranded structure, that is, a short hairpin structure with a 3′-extruding end. Such shRNA, after introduced into cells, is degraded into a form of about 20 bases in length (typically for example, 21, 22 and 23 bases) in the cells, and can inhibit expression of its target gene (MET receptor gene in the present invention) in the same manner as siRNA does. siRNA and shRNA may be any form that can inhibit expression of MET receptor gene. siRNA or shRNA can be artificially produced by chemical synthesis. In vitro production is also possible, and for example, antisense or sense RNA can be produced from template DNA by use of T7 RNA polymerase and T7 promoter. The antisense oligonucleotide may be any nucleotide that is complementary or hybridizable to a contiguous 5- to 100-base sequence in the DNA sequence of MET receptor gene, and such a nucleotide may be DNA or RNA. The antisense oligonucleotide may be modified as far as such modification does not interfere with its functions. The antisense oligonucleotide can be synthesized in a usual manner, and for example, can be easily synthesized with a commercial DNA synthesizer (manufactured by, for example, Applied Biosystems, etc.).

The protein having an HGF-binding domain of a MET receptor extracellular region may be any protein that has an HGF-binding domain of a MET receptor extracellular region and binds to HGF via the domain. Examples thereof include a protein having the whole MET receptor extracellular region, an HGF-binding domain-containing partial protein of the MET receptor extracellular region, and an HGF-binding domain-containing protein having a protein other than the MET receptor extracellular region. The protein having an HGF-binding domain of a MET receptor extracellular region can be obtained by, for example according to a known recombinant technique, constructing an expression vector using a part of the MET receptor-encoding gene, introducing the vector into a suitable host, and collecting and purifying the resulting recombinant protein expressed in the host. For example, in the case of human MET receptor, the extracellular region thereof corresponds a region of the 1st to 932nd amino acid sequence (see SEQ ID NO: 6 and GenBank Accession No. X54559) (Reference: Michieli P, Mazzone M, Basilico C, Cavassa S, Sottile A, Naldini L, Comoglio P M. Targeting the tumor and its microenvironment by a dual-function decoy Met receptor. Cancer Cell. 2004; 6: 61-73).

In the case where the HGF-MET receptor pathway inhibitor is an antibody (for example, an anti-HGF neutralizing antibody, an anti-MET receptor antibody, etc.), the antibody may be a polyclonal antibody or a monoclonal antibody. The antibody also may be a complete antibody molecule or an antibody fragment capable of specifically binding to an antigen (for example, a Fab fragment, a F(ab')₂ fragment, etc.). The polyclonal antibody can be obtained, for example, in the following manner. A mammal (a mouse, a rat, a rabbit, a goat, a horse, etc.) is immunized with an immunogen, i.e. an antigen (for example, IIGF, MET receptor or a fragment thereof) dissolved in PBS, or if needed a mixture thereof with an appropriate amount of a usual adjuvant (for example, Freund's complete adjuvant). The method for immunization is not particularly limited, but preferably, subcutaneous injection or intraperitoneal injection is performed once or several times at appropriate intervals, for example. Then, blood collection from the immunized animal, serum separation and purification from polyclonal antibody fractions are performed in a usual manner, and thus, a polyclonal antibody can be obtained. The monoclonal antibody can be obtained by fusing immune cells (for example, splenocytes) obtained from the above-mentioned immunized mammal with myeloma cells to produce a hybridoma, culturing the hybridoma, and collecting an antibody from the culture. A recombinant monoclonal antibody can be also produced according to recombinant technique, specifically by cloning an antibody gene from the hybridoma, inserting the gene into a suitable vector and introducing the vector into a host.

In the case where antibodies are applied to humans, a chimeric antibody modified so as to have the same constant region as that in a human antibody, and a humanized antibody having a human-derived region except the CDR (complementarity determining region) are preferably used. More preferably, a human monoclonal antibody produced in a transgenic animal such as a transgenic mouse having a human gene involved in antibody production is used. A phage display method can also be used for production of a human antibody. Further, from the thus-obtained antibody, a region for antigen recognition is excised with protease etc. and can be used as Fv, Fab or F(ab′)₂.

In the case where proteins are applied to humans, a human protein is preferably used. The human protein can be produced as a recombinant protein according to a known recombinant technique by use of a human gene encoding the objective protein. For example, the base sequences of human NK4 gene are shown in SEQ ID NOS: 1 and 3, and the amino acid sequences encoded by the base sequences are shown in SEQ ID NOS: 2 and 4, respectively. The base sequence of human MET receptor gene is shown in SEQ ID NO: 5, and the amino acid sequence encoded thereby is shown in SEQ ID NO: 6.

The pharmaceutical composition of the present invention can be appropriately blended with a carrier or an additive usually used in the pharmaceutical field and formulated into a preparation comprising an HGF-MET receptor pathway inhibitor or a pharmaceutically acceptable salt thereof as an active ingredient. Specific examples of the preparation include oral preparations such as tablets, coated tablets, pills, powders, granules, capsules, solutions, suspensions and emulsions; and parenteral preparations such as injections, suppositories, ointments and patches. The blending ratio of the carrier or the additive is appropriately determined based on the range of the blending ratio usually adopted in the pharmaceutical field. The carrier or the additive that can be blended is not particularly limited, and examples thereof include water, physiological saline and other aqueous solvents; various carriers such as aqueous bases and oily bases; and various additives such as excipients, binders, pH adjusters, disintegrants, absorption enhancers, lubricants, colorants, corrigents and fragrances.

Specific examples of such an additive include excipients such as lactose, sucrose, mannitol, sodium chloride, glucose, calcium carbonate, kaolin, crystalline cellulose and silicates; binders such as water, ethanol, simple syrup, glucose in water, a starch solution, a gelatin solution, carboxymethyl cellulose, carboxymethyl cellulose sodium, shellac, methylcellulose, hydroxypropyl methylcellulose, hydroxypropyl cellulose, polyvinyl pyrrolidone, polyvinyl alcohol, gelatin, dextrin and pullulan; pH adjusters such as citric acid, anhydrous citric acid, sodium citrate, sodium citrate dihydrate, disodium hydrogen phosphate anhydrous, sodium dihydrogen phosphate anhydrous, sodium hydrogen phosphate and sodium dihydrogenphosphate; disintegrants such as carmellose calcium, low-substituted hydroxypropyl cellulose, carmellose, croscarmellose sodium, sodium carboxymethyl starch, crospovidone and polysorbate 80; absorption enhancers such as a quaternary ammonium base and sodium lauryl sulfate; lubricants such as purified talc, stearates, polyethylene glycol, colloidal silicic acid and sucrose fatty acid esters; colorants such as yellow iron oxide, yellow iron sesquioxide, iron sesquioxide, β-carotene, titanium oxide, food colors (for example, Food Blue No. 1 etc.), copper chlorophyll and riboflavin; and corrigents such as ascorbic acid, aspartame, sweet hydrangea leaf, sodium chloride, fructose, saccharin and powder sugar.

In the case where the active ingredient in the pharmaceutical composition of the present invention is a nucleic acid (siRNA, shRNA, antisense oligonucleotide, etc.), it can be administered in the form of a non-viral vector or a viral vector. In the case of administration in the form of a non-viral vector, a method in which a nucleic acid molecule is introduced by use of liposome (the liposome method, the HVJ-liposome method, the cationic liposome method, the lipofection method, the lipofectamine method, etc.), microinjection, a method in which a nucleic acid molecule is introduced together with a carrier (metal particles) into cells by use of gene gun, and the like can be used. In the case where siRNA or shRNA is administered to a living body by use of a viral vector, viral vectors such as recombinant adenovirus and retrovirus can be used. Into a DNA or RNA virus such as detoxified retrovirus, adenovirus, adeno-associated virus, herpesvirus, vaccinia virus, poxvirus, poliovirus, Sindbis virus, Sendai virus and SV40, DNA expressing siRNA or shRNA is introduced, and infection with the resulting recombinant virus allows introduction of the objective gene into a cell or a tissue.

The dose of the pharmaceutical composition of the present invention is appropriately determined in consideration of the purpose, the severity of the disease, the age, body weight, sex and medical history of the patient, the kind of the active ingredient, etc. In the case where the subject is an average human weighing about 65 to 70 kg, the daily dose is preferably about 0.05 to 2000 mg, and more preferably about 0.1 to 200 mg. The daily total dose may be a single dose or may be divided into several portions.

The cancer therapeutic agent of the present invention comprises a molecular target drug in combination with an HGF-MET receptor pathway inhibitor, and for example, preferably comprises a molecular target drug in combination with the above-mentioned pharmaceutical composition of the present invention. The HGF-MET receptor pathway inhibitor and the molecular target drug may be simultaneously administered. Alternatively, the HGF-MET receptor pathway inhibitor and the molecular target drug may be successively administered in this order, and vice versa. Further, the HGF-MET receptor pathway inhibitor and the molecular target drug may be separately administered in this order at intervals, and vice versa. The order and interval of administration can be appropriately selected depending on the preparation comprising an HGF-MET receptor pathway inhibitor, the molecular target drug used in combination therewith, the kind of cancer cells to be treated, the patient's condition, etc. Here, “simultaneously administered” means that plural drugs are administered at almost the same time. “Separately administered” means that plural drugs are administered at different times, and for example, means such a case that a drug is administered on the first day and another drug is administered on the second day. “Successively administered” means that plural drugs are administered in a certain order, and for example, means such a case that a drug is first administered and, after a certain period, another drug is administered.

Regarding a cancer which is a target of treatment with a molecular target drug but is refractory or less sensitive to the molecular target drug, the cancer therapeutic agent of the present invention enhances the sensitivity of the cancer to the molecular target drug and makes the molecular target drug exert an effect on the cancer, and therefore extremely efficient treatment of the cancer is made possible. In conventional treatment, even though a molecular target drug is effective, the sensitivity to the molecular target drug is often reduced by some factors and thus such effectiveness that the cancer regresses remarkably or disappears almost completely cannot be expected. On the other hand, the cancer therapeutic agent of the present invention is so therapeutically effective that the cancer regresses remarkably or disappears almost completely. In conventional treatment, even though the effect of the molecular target drug continues during a certain period, the sensitivity to the molecular target drug reduces shortly or, in many cases, within a year after the treatment starts and thus life-prolonging effect that fulfills the patient's expectation cannot be achieved. On the other hand, the cancer therapeutic agent of the present invention, since it is so therapeutically effective that the cancer regresses remarkably or disappears almost completely, can bring remarkable life extension or complete cure. Accordingly, the present invention is beneficial for cancer patients, and its social significance is extremely great.

Combining a molecular target drug which constitutes the cancer therapeutic agent of the present invention, and a preparation comprising an HGF-MET receptor pathway inhibitor into the form of a kit is also included in the scope of the present invention. The kit of the present invention comprises a means to separately hold a molecular target drug and a preparation comprising an HGF-MET receptor pathway inhibitor, such as a divided container, a divided bottle and a divided foil packet. The kit of the present invention is suitable when separate compositions are administered in different dosage forms at different dosage intervals. The kit usually comprises an instruction manual for administration, and the instruction manual may be written or printed on paper or other media, or may be provided in the form of electronic media, such as magnetic tape, computer-readable disk and CD-ROM.

### EXAMPLES

Hereinafter, the present invention will be illustrated in detail by examples, but is not limited thereto.

### Example 1

### HGF-Induced Gefitinib Hyposensitivity of Human Non-Small-Cell Lung Cancer Cell Line with Egfr-Activating Mutation

### (1) Experimental Materials

Human non-small-cell lung cancer cell lines PC-9 (hereinafter referred to as “PC-9”) and HCC827 (hereinafter referred to as “HCC827”) were used. These cell lines express mutant EGFR having a deletion of residues 746(E) to 750(A) in the amino acid sequence of EGFR, and are sensitive to gefitinib. Neither of the cell lines is accompanied by new EGFR mutation associated with gefitinib resistance (hyposensitivity) or by amplification of MET receptor gene associated therewith. PC-9 and HCC827 were purchased from Immuno-Biological Laboratories Co. and ATCC, respectively. An RPMI1640 culture medium supplemented with 10% FBS, penicillin (100 units/mL), streptomycin (100 units/mL) and glutamine (2 mmol/L) was used for culture of PC-9 and HCC827.

Gefitinib was obtained from AstraZeneca. HGF (recombinant human HGF protein) was prepared according to the description in “Kato S, Funakoshi H, Nakamura T, et al. Acta Neuropathol. 2003 August; 106(2): 112-20”. EGF and IGF-I were purchased from Invitrogen. TGF-α was purchased from Biosource. An anti-human HGF neutralizing antibody (goat) and a control IgG (goat) were purchased from R&D System.

### (2) Experimental Methods

### (a) Induction of Gefitinib Resistance (Hyposensitivity) by HGF

PC-9 or HCC827 was seeded at 2×10³ cells/well on 96-well plates and cultured for 24 hours. After that, gefitinib and HGF were added to the wells in combinations thereof at different concentrations. Specifically, gefitinib was added to each well so that the final concentration might be set to the five levels of 0.01 to 1 μM. Wells without gefitinib were also prepared for this experiment. An HGF solution was added to the wells so that the final concentration might be set to the five levels of 2 to 50 ng/mL. Wells without HGF were also prepared for this experiment. After 72-hour culture, 50 μL of an MTT solution (2 mg/mL, manufactured by Sigma) was added and incubation was performed at 37° C. for 2 hours. The culture media were removed, and dark blue crystals were dissolved by adding 100 μL of DMSO. The absorbance was measured with a microplate reader MTP-120 (Corona Electric Co., Ltd.) at the detection and reference wavelengths of 550 nm and 630 nm, respectively. The growth rate was shown as a relative value based on an untreated control. Each experiment was performed in triplicate and repeated thrice independently.

(b) Examination on Induction of Gefitinib Resistance (Hyposensitivity) of HCC827 by HGF Pretreated with Anti-Human HGF Neutralizing Antibody

An HGF solution or a vehicle without HGF (control) was pretreated with the anti-human HGF neutralizing antibody or the control IgG at 37° C. for 1 hour. In the same manner as in the above (a), HCC827 was seeded at 2×10³ cells/well on 96-well plates, and after 24-hour culture, gefitinib was added to the wells so that the final concentration might be 0.3 μM. To the wells with or without gefitinib, each of the pretreated solutions was added and then culture was continued for 72 hours. The final concentrations of HGF, the anti-human HGF neutralizing antibody and the control IgG in the culture media were 20 ng/mL, 2 μg/mL and 2 μg/mL, respectively. After 72-hour culture, the degree of cell growth was measured by the MTT method and the growth rate was calculated in the same manner as in the above (a).

### (c) Examination on Induction of Gefitinib Resistance (Hyposensitivity) by Various Growth Factors

In the same manner as in the above (a), PC-9 or HCC827 was seeded at 2×10³ cells/well on 96-well plates, and after 24-hour culture, gefitinib was added to the wells so that the final concentration might be 0.3 or 1 μM. HGF, EGF, TGF-α or IGF-I was further added to give a final concentration of 20 ng/mL and then culture was continued for 72 hours. Wells without gefitinib and wells into which a culture medium was added instead of the growth factors were prepared for this experiment. After 72-hour culture, the degree of cell growth was measured by the MTT method and the growth rate was calculated in the same manner as in the above (a).

### (3) Results

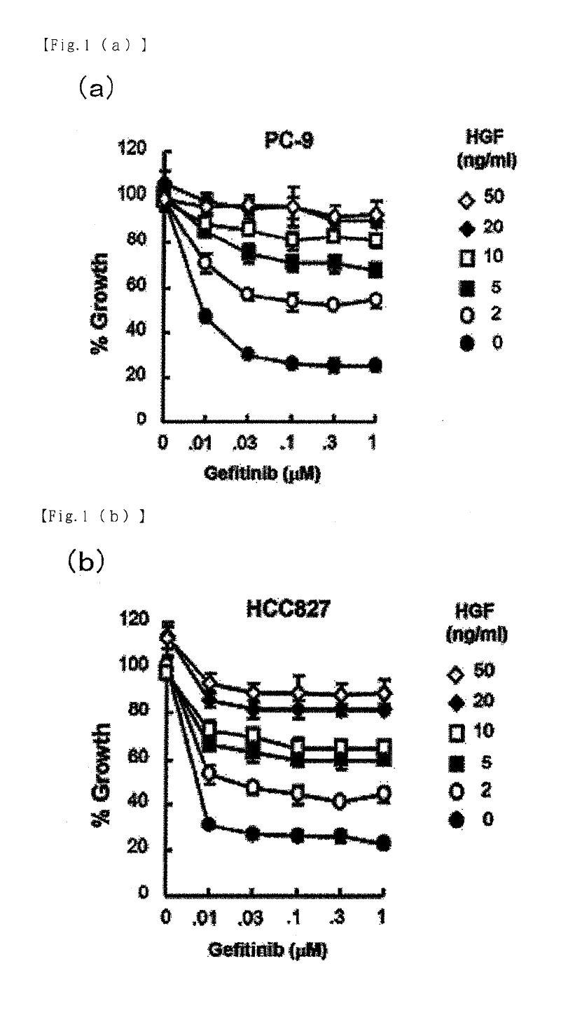

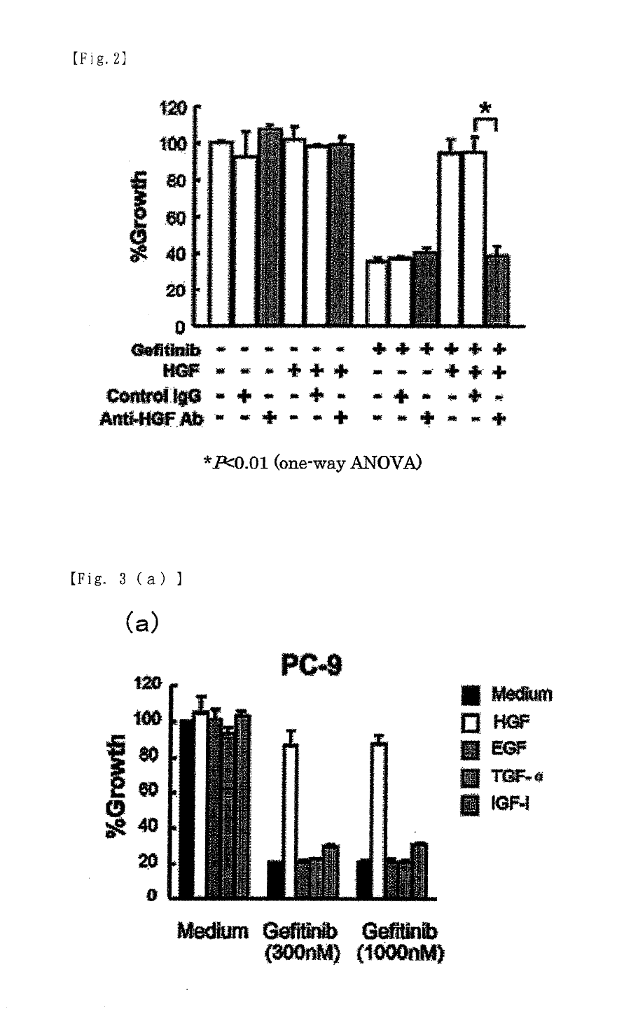

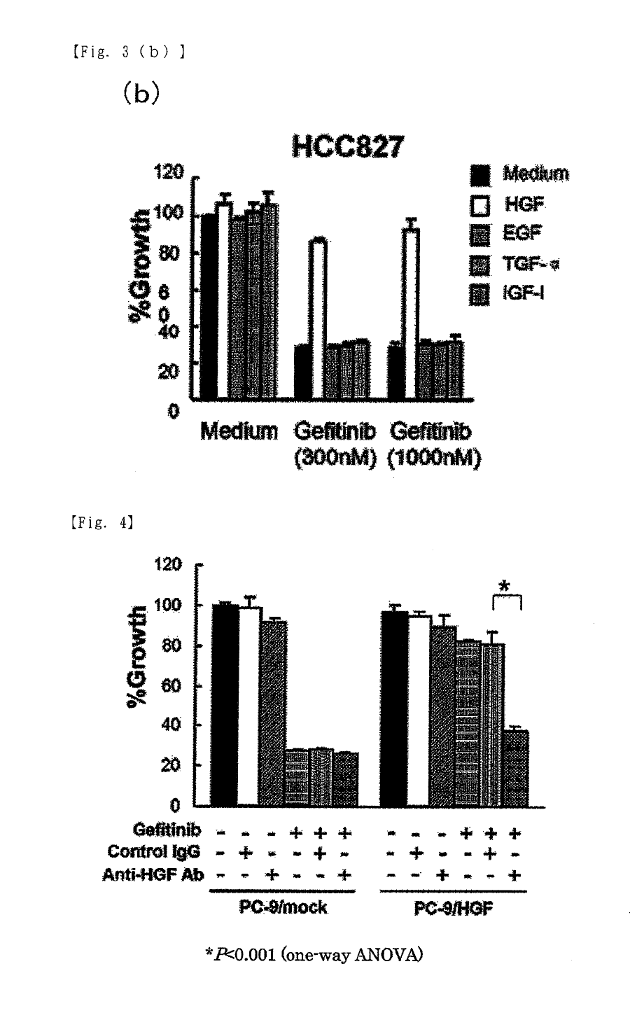

The results of (a) are shown in FIGS. 1(a) and 1(b), the results of (b) are shown in FIG. 2, and the results of (c) are shown in FIGS. 3(a) and 3(b).

FIGS. 1(a) and 1(b) clearly show that HGF induced gefitinib resistance of PC-9 and HCC827 in a concentration-dependent manner. As is clear from FIG. 2, although HGF pretreated with the anti-HGF neutralizing antibody lost the capability of inducing gefitinib resistance of HCC827, HGF pretreated with the control IgG maintained the capability of inducing gefitinib resistance of HCC827. In addition, FIGS. 3(a) and 3(b) clearly show that the growth factors (EGF, TGF-α, IGF-I) except HGF slightly induced gefitinib resistance while HGF had a remarkable capability of inducing gefitinib resistance.

These results demonstrated that, even though human non-small-cell lung cancer cells are originally sensitive to gefitinib and are not accompanied by new EGFR mutation associated with gefitinib resistance or by amplification of MET receptor gene associated therewith, HGF reduces the sensitivity, i.e., develops the resistance to gefitinib.

### Example 2

### Examination on PC-9 Transfected with HGF Expression Vector

### (1) Experimental Materials

The cells used were the same PC-9 as that in Example 1. The gefitinib, anti-human HGF neutralizing antibody and control IgG used were the same as those in Example 1.

### (2) Experimental Methods

An HGF expression vector was prepared according to the description in “Ueki T, Kaneda Y, Tsutsui H, et al. Hepatocyte growth factor gene therapy of liver cirrhosis in rats. Nature Medicine 5, 226-230 (1 Feb. 1999)”. On the day before transfection, PC-9 prepared in a culture medium without any antibiotics was seeded at 2×10⁴ cells/400 μL on 24-well plates and then cultured for 24 hours. The HGF expression vector was transfected into the cells by use of Lipofectamine 2000 (1 μL) (PC-9/HGF). As a control, a vector without the HGF gene was similarly transfected into the cells (PC-9/mock). After 24-hour culture, the cells were washed with PBS. Then, 0.3 μM of gefitinib (final concentration), 2 μg/mL of the anti-human HGF neutralizing antibody (final concentration) and/or 2 μg/mL of the control IgG (final concentration) were added, or not added, and then culture was continued for 72 hours. After 72-hour culture, the degree of cell growth was measured by the MTT method and the growth rate was calculated in the same manner as in Example 1.

The results are shown in FIG. 4. As is clear from FIG. 4, PC-9/mock was sensitive to gefitinib while PC-9/HGF was less sensitive to gefitinib and thus had an acquired resistance thereto. When the anti-human HGF neutralizing antibody was added to PC-9/HGF, such a reduction in sensitivity was significantly inhibited.

These results demonstrated the following: cells become capable of producing HGF due to HGF gene expression and also acquire the resistance (namely, reduced sensitivity) to gefitinib; and this resistance to gefitinib is inhibited by neutralization of HGF activity, i.e., inhibition of HGF-MET receptor pathway, and in other words, HGF-induced resistance to gefitinib is inhibited by inhibition of HGF-MET receptor pathway.

### Example 3

### Examination on Effects of HGF-MET Receptor Pathway Inhibitor on Fibroblast-Derived-HGF-Induced Gefitinib Resistance of Non-Small-Cell Lung Cancer Cell

### (1) Experimental Materials

The cells used were the same PC-9 as that in Example 1 and normal human embryonic lung fibroblast MRC-5 (hereinafter referred to as “MRC-5”). MRC-5 is available from, for example, ATCC (ATCC No. CCL-171). An RPMI1640 culture medium supplemented with 10% FBS, penicillin (100 units/mL), streptomycin (100 units/mL) and glutamine (2 mmol/L) was used for culture of PC-9. A DMEM culture medium supplemented with 10% FBS, penicillin (100 units/mL), streptomycin (100 units/mL) and glutamine (2 mmol/L) was used for culture of MRC-5.

Gefitinib was obtained from AstraZeneca. HGF (recombinant human HGF protein) and NK4 (recombinant human NK4 protein) were obtained from Kringle Pharma, Inc. An anti-human HGF neutralizing antibody (goat) and a control IgG (goat) for culture cell experiments were purchased from R&D System. An anti-human HGF neutralizing antibody (immunoglobulin) for animal experiments was purified by chromatography on a protein A column from antiserum obtained from a rabbit into which human HGF protein had been administered. Gefitinib was suspended in a water containing 1% Tween80 so that the concentration might be 2.5 mg/mL, and this suspension was used for administration. NK4 and the anti-human HGF neutralizing antibody were separately prepared in physiological saline (Otsuka Pharmaceutical Factory, Inc.) at the concentrations of 1.13 mg/mL and 1.0 mg/mL, respectively, and these solutions were used for administration.

### (2) Experimental Methods

### (a) Induction of Gefitinib Resistance of PC-9 by MRC-5-Derived HGF and Effects of Anti-HGF Neutralizing Antibody

In the case of coculture of PC-9 (lung cancer cells) with MRC-5 (fibroblasts), a transwell chamber (24 wells, Coster) was used. PC-9 was seeded in the lower wells at 8×10³ cells/700 μL and MRC-5 was seeded in the upper wells at 1×10⁴ cells/300 μL, and then culture was performed for 24 hours. After that, 0.3 μM of gefitinib (final concentration) was added or not added, 2 μg/mL of the control IgG (final concentration) or 2 μg/mL of the anti-human HGF neutralizing antibody (final concentration) was added or not added, and then culture was continued for 72 hours. After 72-hour culture, the degree of growth of PC-9 in the lower wells was measured by the MTT method and the growth rate was calculated in the same manner as in Example 1.

In the case of single culture of PC-9, PC-9 was seeded in each well of 24-well plates at 8×10³ cells/700 μL and then cultured for 24 hours. After that, 50 ng/mL of HGF (final concentration) was added or not added, 0.3 μM of gefitinib (final concentration) was added or not added, 2 μg/mL of the control IgG (final concentration) or 2 μg/mL of the anti-human HGF neutralizing antibody (final concentration) was added or not added, and then culture was continued for 72 hours. After 72-hour culture, the degree of cell growth was measured by the MTT method and the growth rate was calculated in the same manner as in Example 1.

(b) Effects of Anti-HGF Neutralizing Antibody and NK4 on Xenograft Tumor Transplanted into SCID Mice

SCID mice (female, 5 weeks old) were purchased from CLEA Japan.

100 μL of a cell suspension containing PC-9 (5×10⁶ cells) and MRC-5 (5×10⁶ cells) was subcutaneously inoculated from the back skin of each SCID mouse. Four days later, mice bearing a tumor exceeding 4 mm in diameter were randomly divided into 6 groups (5 mice per group) as shown in Table 1.

TABLE 1

| | | anti-HGF | | |
|---|---|---|---|---|
| Group | | neutralizing | | Legend |
| No. | Gefitinib | antibody | NK4 | in FIG. 1 |
| 1 | − | − | − | Control |
| 2 | − | + | − | HGF Ab |
| 3 | − | − | + | NK4 |
| 4 | + | − | − | Gefitinib |
| 5 | + | + | − | HGF |
| | | | | Ab + Gefitinib |
| 6 | + | − | + | NK4 + Gefitinib |

In gefitinib-treated groups (groups 4, 5 and 6 in Table 1), gefitinib (25 mg/kg/day) was orally administered once daily in the morning for 13 days from 4 days to 16 days after cell inoculation. In gefitinib-non-treated groups (groups 1, 2 and 3 in Table 1), a water containing 1% Tween80 was orally administered once daily in a similar manner to the above. In anti-HGF neutralizing antibody-treated groups (groups 2 and 5 in Table 1), the anti-HGF neutralizing antibody (5 mg/kg/day) was intraperitoneally administered once daily immediately after gefitinib administration for 13 days from 4 days to 16 days after cell inoculation. In NK4-treated groups (groups 3 and 6 in Table 1), NK4 (9 mg/kg/day) was intraperitoneally administered in divided portions twice a day in the morning immediately after gefitinib administration and in the evening for 13 days from 4 days to 16 days after cell inoculation.

The width and length of tumor were measured every other day, and the tumor area (width×length) was calculated. This experiment was conducted according to the United Kingdom Coordinating Committee on Cancer Research Guidelines for the Welfare of Animals in Experimental Neoplasia.

### (3) Results

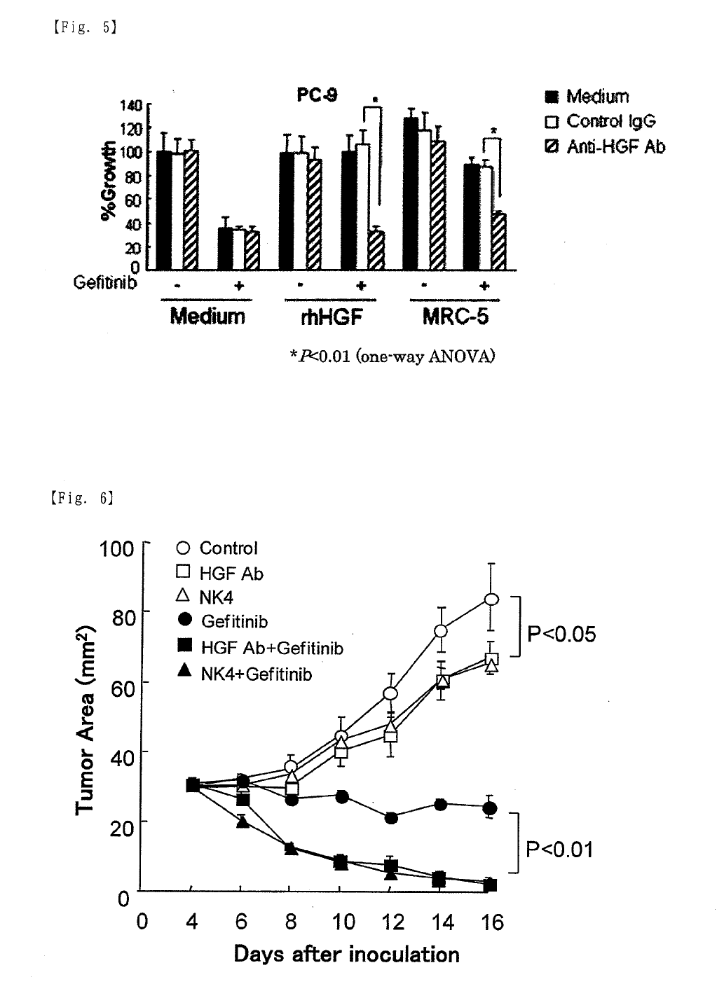

The results of (a) are shown in FIG. 5. In FIG. 5, “Medium” represents growth of PC-9 cultured in culture media without HGF, “rhHGF” represents growth of PC-9 cultured in culture media with HGF (recombinant human HGF), and “MRC-5” represents growth of PC-9 cocultured with MRC-5 in culture media without HGF. As shown in FIG. 5, in the case where PC-9 was cocultured with MRC-5, HGF produced by MRC-5 induced gefitinib resistance (hyposensitivity) of PC-9 in the same manner as in the case where rhHGF was added to culture media, and this gefitinib resistance was inhibited by the anti-human HGF antibody.

The results of (b) are shown in FIG. 6. FIG. 6 clearly shows that, in the case where the vehicle alone was orally administered (control group, “Control” in FIG. 6), the tumor volume increased with time until 16 days after cell inoculation. In combined administration of the vehicle and the anti-HGF neutralizing antibody (“HGF Ab” in FIG. 6) and combined administration of the vehicle and NK4 (“NK4” in FIG. 6), the tumor volume increased with time, but the tumor growth rate was slightly reduced as compared with that of the control group. In the case where gefitinib alone was orally administered (“Gefitinib” in FIG. 6), increase in tumor volume was inhibited, but no significant regression was observed. These results demonstrated that the formed tumor was less sensitive to gefitinib. On the other hand, in combined administration of the anti-HGF neutralizing antibody and gefitinib (“HGF Ab+Gefitinib” in FIG. 6), and combined administration of NK4 and gefitinib (“NK4+Gefitinib” in FIG. 6), the tumor significantly regressed, and at 16 days after cell inoculation, almost completely disappeared (the tumor volume was reduced to approximately 0).

### Example 4

### Measurement of HGF Concentration in Xenograft Tumor Transplanted into SCID Mice

In the same manner as in Example 3, 100 μL of a cell suspension containing PC-9 (5×10⁶ cells) and MRC-5 (5×10⁶ cells) was subcutaneously inoculated from the back skin of each SCID mouse, and after 4 days, tumor tissue was excised. As a control, PC-9 alone was inoculated similarly, and after 4 days, tumor tissue was excised. After the tumor tissue was homogenized in a protease inhibitor cocktail (20 mM Tris-HCl (pH 7.5), 2 M NaCl, 0.1% Tween-80, 2 mM EDTA, 1 mM PMSF), the homogenate was centrifuged at 12,000×g for 30 minutes. The supernatant was collected as an extract solution and human HGF in the extract solution was quantified by use of an ELISA kit (IMMUNIS HGF EIA, Institute of Immunology).

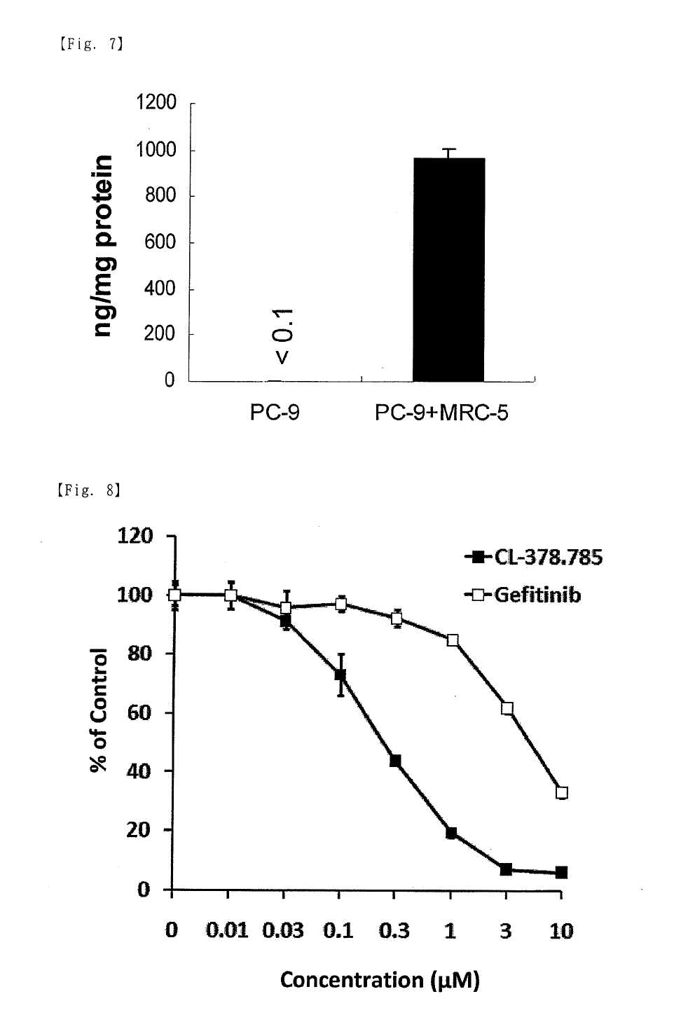

The results are shown in FIG. 7. As is clear from FIG. 7, HGF was not detected in the extract solution from the tumor tissue formed of PC-9 alone, but was detected at a high level in the extract solution from the tumor tissue formed of the mixture of PC-9 and MRC-5 cells. These results demonstrated that inoculation of a mixture of gefitinib-sensitive human non-small-cell lung cancer cell line PC-9 with normal fibroblast MRC-5 leads to production of HGF and induction of gefitinib resistance of PC-9.

### Example 5

### Examination on Effects on Cancer Cells Resistant to Irreversible EGFR Inhibitor

### (1) Experimental Materials

A human non-small-cell lung cancer cell line H1975 (hereinafter referred to as “H1975”), which is resistant to gefitinib and sensitive to CL-387,785, was used. H1975 is available from, for example, ATCC (ATCC No. CRL-5908). An RPMI1640 culture medium supplemented with 10% FBS, penicillin (100 units/mL), streptomycin (100 units/mL) and glutamine (2 mmol/L) was used for culture of H1975.

Gefitinib was obtained from AstraZeneca. CL-387,785 was obtained from COSMO BIO. HGF (recombinant human HGF protein) and NK4 (recombinant human NK4 protein) were obtained from Kringle Pharma, Inc. An anti-human HGF neutralizing antibody (Lot No. ALP01) was purchased from R&D Systems.

### (2) Experimental Methods

### (a) Effects of Gefitinib or CL-387,785 on H1975

After 80% confluent H1975 was stripped and collected, the cells were seeded at 2×10³ cells/well on 96-well plates and cultured for 24 hours. After that, gefitinib or CL-387,785 was added to each well so that the final concentration might be set to the seven levels of 0.01 to 10 μM, and then culture was continued for 72 hours. After that, 50 μl, of an MTT solution (2 mg/mL, manufactured by Sigma) was added and incubation was performed at 37° C. for 2 hours. The culture media were removed, and dark blue crystals were dissolved by adding 100 μL of DMSO. The absorbance was measured with a microplate reader MTP-120 (Corona Electric Co., Ltd.) at the detection and reference wavelengths of 550 nm and 630 nm, respectively. The growth rate was shown as a relative value based on an untreated control. Each experiment was performed in triplicate and repeated thrice independently.

### (b) Induction of CL-387, 785 Resistance (Hyposensitivity) by HGF

In the same manner as in the above (a), H1975 was seeded at 2×10³ cells/well on 96-well plates. After 24-hour culture, CL-387,785 was added to the wells so that the final concentration might be set to the five levels of 0.03 to 3 μM. An HGF solution was further added to give a final concentration of 50 ng/mL, or was not added, and then culture was continued for 72 hours. The degree of cell growth was measured by the MTT method and the growth rate was calculated in the same manner as in the above (a).

(c) Effects of Anti-Human HGF Neutralizing Antibody or NK4 on H1975 with HGF-Induced CL-387,785-Resistance

In the same manner as in the above (a), H1975 was seeded at 2×10³ cells/well on 96-well plates. After 24-hour culture, 0.3 μM of CL-387,785 (final concentration), 50 ng/mL of HGF (final concentration), 2 μg/mL of the anti-human HGF neutralizing antibody (final concentration), and/or 0.3 μM of NK4 (final concentration) were added as shown in Table 2, and then culture was continued for 72 hours. The degree of cell growth was measured by the MTT method and the growth rate was calculated in the same manner as in the above (a).

TABLE 2

| | | | anti-HGF | |
|---|---|---|---|---|
| | | | neutralizing | |
| | CL387,785 | HGF | antibody | NK4 |
| Control | − | − | − | − |
| | − | − | + | − |
| | − | − | − | + |
| CL387,785 | + | − | − | − |
| | + | − | + | − |
| | + | − | − | + |
| CL387,785 + | + | + | − | − |
| HGF | + | + | + | − |
| | + | + | − | + |

### (3) Results

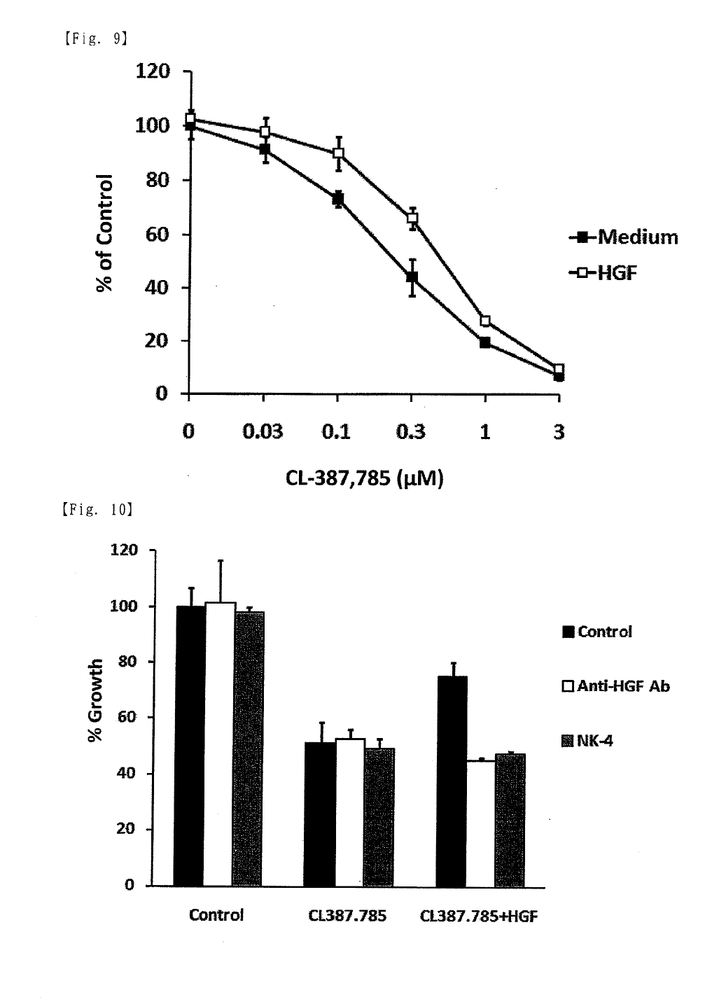

The results of (a) are shown in FIG. 8, the results of (b) are shown in FIG. 9, and the results of (c) are shown in FIG. 10. As shown in FIG. 8, H1975 was resistant to gefitinib and sensitive to CL-387,785. FIG. 9 shows that addition of HGF to the culture medium reduced the sensitivity of H1975 to CL-387,785. FIG. 10 clearly shows that the anti-human HGF neutralizing antibody or NK4 inhibited the HGF-induced hyposensitivity of H1975 to CL-387,785. These results demonstrated that, even though the sensitivity of H1975 cells to CL-387,785 is reduced by the action of HGF, an anti-human HGF neutralizing antibody or NK4 overcomes such reduction in sensitivity.

### Example 6

### Examination on Effects of MET Receptor Tyrosine Kinase Inhibitor SU11274 on HGF-Induced Resistance of Non-Small-Cell Lung Cancer Cells to Molecular Target Drug

### (1) Experimental Materials

The cells used were PC-9 or H1975. An RPMI1640 culture medium supplemented with 10% FBS, penicillin (100 units/mL), streptomycin (100 units/mL) and glutamine (2 mmol/L) was used for culture of PC-9 and H1975.

Gefitinib was obtained from AstraZeneca. CL-387,785 was obtained from COSMO BIO. HGF (recombinant human HGF protein) and NK4 (recombinant human NK4 protein) were obtained from Kringle Pharma, Inc. An anti-human HGF neutralizing antibody (goat) was purchased from R&D System. SU11274 was obtained from Calbiochem.

### (2) Experimental Methods

(a) Effects of SU11274 on PC-9 with HGF-Induced Gefitinib-Resistance

PC-9 was seeded at 2×10³ cells/well on 96-well plates and cultured for 24 hours. After that, 20 ng/mL of HGF (final concentration), 0.3 μM of gefitinib (final concentration), 1 μg/mL of the anti-human HGF neutralizing antibody (final concentration), 0.3 μM of NK4 (final concentration), and/or 0.3 μM of SU11274 (final concentration) were added as shown in Table 3, and then culture was continued for 72 hours. After that, 50 μL of an MTT solution (2 mg/mL, manufactured by Sigma) was added and incubation was performed at 37° C. for 2 hours. The culture media were removed, and dark blue crystals were dissolved by adding 100 μL of DMSO. The absorbance was measured with a microplate reader MTP-120 (Corona Electric Co., Ltd.) at the detection and reference wavelengths of 550 nm and 630 nm, respectively. The growth rate was shown as a relative value based on an untreated control. Each experiment was performed in triplicate and repeated thrice independently.

TABLE 3

| | | | anti-HGF | | |
|---|---|---|---|---|---|
| | | | neutralizing | | |
| | HGF | Gefitinib | antibody | NK4 | SU11274 |
| Medium | − | − | − | − | − |
| | − | − | + | − | − |
| | − | − | − | + | − |
| | − | − | − | − | + |
| HGF | + | − | − | − | − |
| | + | − | + | − | − |
| | + | − | − | + | − |
| | + | − | − | − | + |
| Gefitinib | − | + | − | − | − |
| | − | + | + | − | − |
| | − | + | − | + | − |
| | − | + | − | − | + |
| Gefitinib + | + | + | − | − | − |
| HGF | + | + | + | − | − |
| | + | + | − | + | − |
| | + | + | − | − | + |

(b) Effects of SU11274 on H1975 with HGF-Induced CL-387,785-Resistance

H1975 was seeded at 2×10³ cells/well on 96-well plates and cultured for 24 hours. After that, 0.3 μM of CL-387,785 (final concentration), 50 ng/mL of HGF (final concentration), 2 μg/mL of the anti-human HGF neutralizing antibody (final concentration), 0.3 μM of NK4 (final concentration), and/or 1 NM of SU11274 (final concentration) were added as shown in Table 4, and then culture was continued for 72 hours. The degree of cell growth was measured by the MTT method and the growth rate was calculated in the same manner as in the above (a).

TABLE 4

| | | | anti-HGF | | |
|---|---|---|---|---|---|
| | | | neutralizing | | |
| | CL387,785 | HGF | antibody | NK4 | SU11274 |
| Medium | − | − | − | − | − |
| | − | − | + | − | − |
| | − | − | − | + | − |
| | − | − | − | − | + |
| CL387,785 | + | − | − | − | − |
| | + | − | + | − | − |
| | + | − | − | + | − |
| | + | − | − | − | + |
| CL387,785 + | + | + | − | − | − |
| HGF | + | + | + | − | − |
| | + | + | − | + | − |
| | + | + | − | − | + |

### (3) Results

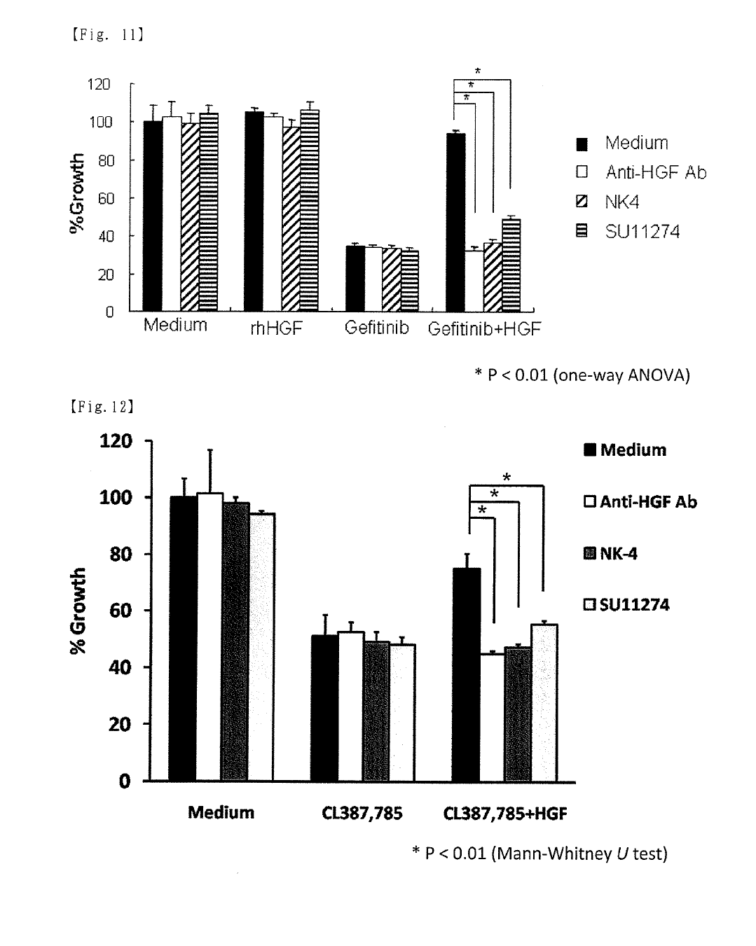

The results of (a) are shown in FIG. 11 and the results of (b) are shown in FIG. 12. FIG. 11 clearly shows that, like the anti-human HGF neutralizing antibody and NK4, SU11274 significantly inhibited HGF-induced hyposensitivity of PC-9 to gefitinib. FIG. 12 clearly shows that, like the anti-human HGF neutralizing antibody and NK4, SU11274 significantly inhibited HGF-induced hyposensitivity of H1975 to CL-387,785.

The present invention is not limited to the aforementioned embodiments and examples, and various modifications can be made within the scope of the appended claims. Other embodiments obtainable by suitably combining technical means disclosed in different embodiments of the present invention are also included in the technical scope of the present invention. All the academic publications and patent literature cited in the above description are incorporated herein by reference.

1. A pharmaceutical composition comprising an HGF-MET receptor pathway inhibitor, which enhances the sensitivity of a cancer to a molecular target drug, wherein the cancer is a target of treatment with the molecular target drug but is refractory to the molecular target drug or less sensitive to the molecular target drug.

2. The pharmaceutical composition according to claim 1, wherein the cancer refractory to the molecular target drug or less sensitive to the molecular target drug is not accompanied by amplification of MET receptor gene.

3. The pharmaceutical composition according to claim 1, wherein the molecular target drug is an EGFR tyrosine kinase inhibitor.

4. The pharmaceutical composition according to claim 3, wherein the EGFR tyrosine kinase inhibitor is a reversible EGFR tyrosine kinase inhibitor.

5. The pharmaceutical composition according to claim 3, wherein the EGFR tyrosine kinase inhibitor is an irreversible EGFR tyrosine kinase inhibitor.

6. The pharmaceutical composition according to claim 1, wherein the HGF-MET receptor pathway inhibitor is one or more kinds selected from the group consisting of an anti-HGF neutralizing antibody, NK4, a MET receptor tyrosine kinase inhibitor, an anti-MET receptor antibody, a MET receptor expression inhibitor and a protein having an HGF-binding domain of a MET receptor extracellular region.

7. The pharmaceutical composition according to claim 1, wherein the cancer being a target of treatment with the molecular target drug is lung cancer, breast cancer, colon cancer, prostate cancer, brain tumor, pancreatic cancer, gallbladder cancer, renal cancer, chronic myelogenous leukemia, gastrointestinal stromal tumor, esophageal cancer, head-and-neck tumor or gastric cancer.

8. A cancer therapeutic agent comprising a molecular target drug in combination with an HGF-MET receptor pathway inhibitor, wherein the cancer is a target of treatment with the molecular target drug but is refractory to the molecular target drug or less sensitive to the molecular target drug.

9. The cancer therapeutic agent according to claim 8, wherein the cancer refractory to the molecular target drug or less sensitive to the molecular target drug is not accompanied by amplification of MET receptor gene.

10. The cancer therapeutic agent according to claim 8, wherein the molecular target drug is an EGFR tyrosine kinase inhibitor.

11. The cancer therapeutic agent according to claim 10, wherein the EGFR tyrosine kinase inhibitor is a reversible EGFR tyrosine kinase inhibitor.

12. The cancer therapeutic agent according to claim 10, wherein the EGFR tyrosine kinase inhibitor is an irreversible EGFR tyrosine kinase inhibitor.

13. The cancer therapeutic agent according to claim 8, wherein the HGF-MET receptor pathway inhibitor is one or more kinds selected from the group consisting of an anti-HGF neutralizing antibody, NK4, a MET receptor tyrosine kinase inhibitor, an anti-MET receptor antibody, a MET receptor expression inhibitor and a protein having an HGF-binding domain of a MET receptor extracellular region.

14. The cancer therapeutic agent according to claim 8, wherein the cancer being a target of treatment with the molecular target drug is lung cancer, breast cancer, colon cancer, prostate cancer, brain tumor, pancreatic cancer, gallbladder cancer, renal cancer, chronic myelogenous leukemia, gastrointestinal stromal tumor, esophageal cancer, head-and-neck tumor or gastric cancer.
